(19) United States
(12) Patent Application Publication
(10) Pub. No.: US 2025/0195261 A1
(43) Pub. Date: Jun. 19, 2025

(54) METHOD AND APPARATUS FOR ANTI-SNORE RING

(71) Applicant: Saj Muzaffar, Hounslow (GB)

(72) Inventor: Saj Muzaffar, Hounslow (GB)

(21) Appl. No.: 18/925,135

(22) Filed: Oct. 24, 2024

(30) Foreign Application Priority Data
Dec. 19, 2023 (GB) 2319513.4

(51) Int. Cl. A61F 5/56; A61B 7/00

(52) U.S. Cl. CPC A61F 5/56; A61B 7/003

(57) ABSTRACT

A wearable anti-snore device in the form of a flexible finger ring. It employs a snoring detection system that alerts users to snoring incidents during sleep using gentle vibrations. The device connects to smartphones or tablets via Bluetooth or Wi-Fi, allowing for detailed sleep monitoring and analysis. Customizable features enhance user comfort and detection accuracy. An independent version operates with integrated microphone and electronics.

### CROSS REFERENCE TO RELATED APPLICATIONS

This application claims priority to United Kingdom Patent Application Number GB 2319513.4, filed Dec. 19, 2023, the disclosure of which is incorporated by reference in its entirety.

### FIELD OF THE INVENTION

This invention introduces a discreet and user-friendly wearable anti-snoring device, taking the form of a flexible finger ring. Employing a sophisticated snoring detection system, this device utilizes subtle vibrations to alert users to snoring incidents during sleep. It seamlessly integrates with a mobile application on smartphones or tablets through Bluetooth Low Energy (BLE) or Wi-Fi, enabling comprehensive sleep monitoring and analysis. This innovative wearable invention focuses on sleep monitoring and snore detection, addressing the widespread issue of snoring that adversely affects sleep quality.

### BACKGROUND OF THE INVENTION

Snoring, a common disruptor of sleep, necessitates a discreet and efficient solution.

### SUMMARY OF THE INVENTION

This invention provides this solution through a flexible ring designed for active detection and resolution of snoring disturbances during sleep. Upon detecting snoring patterns, sounds, the ring initiates a gentle vibration, prompting the wearer to adjust their sleeping position, effectively mitigating snoring. The device's soft and pliable structure, crafted from silicon or similar materials, ensures one size fits all and comfort during sleep.

In one embodiment of the invention, the snoring detection system is configured to recognize and analyze snoring patterns and sounds. This may be by means of hardware, software, firmware, or a combination thereof.

In another embodiment of the invention, the device is a flexible ring, utilizing materials such as silicon, the device ensures a secure and comfortable fit for users of varying finger sizes. An adjustable opening enhances usability.

In another embodiment of the invention the snoring detection system may employ cutting-edge Ai (machine learning) software and algorithms to continuously learn user-specific snoring behaviors to enhance detection accuracy.

In another embodiment of the invention a smartphone detects snoring sounds via its microphone via a mobile application that conducts in-depth analysis of snoring and if snoring is detected, it transmits a signal to the ring to induce vibration. The connectivity between the ring and the smartphone may be by means of Bluetooth Low Energy (BLE) or Wi-Fi, allowing real-time monitoring and data analysis through a dedicated mobile application that may also enhance the user insight by charts and the like.

The said smartphone application may allow users to customize the intensity, duration, and frequency of vibrations, as well as the length of snores, pauses between snores, and the number of snores to detect. This customization aims to enhance snore detection accuracy and user comfort.

In another embodiment of the invention the device operates independently of a smartphone, featuring integrated microphone and electronics for stand-alone snoring detection. The device may be powered by a compact, energy-efficient battery. The battery may be disposable, or it may be rechargeable. The device may be equipped with the capability to enter a deep sleep or ultra-low-power mode, ensuring efficient conservation of battery power.

Other aspects of the disclosed invention will become apparent from the following detailed description, the accompanying drawings and the appended claims.

### BRIEF DESCRIPTION OF THE DRAWINGS

FIG. 1 illustrates the adjustable opening to enhance usability of the ring to ensure a secure and comfortable fit for users of varying finger sizes.

FIG. 2 illustrates a schematic representation of one embodiment of the anti-snore ring apparatus as per the invention.

FIG. 3 illustrates a schematic representation of another embodiment of the anti-snore ring apparatus as per the invention.

FIG. 4 illustrates is a flow diagram of a method for anti-snore detection and intervention according to the present invention.

### DETAILED DESCRIPTION OF THE INVENTION

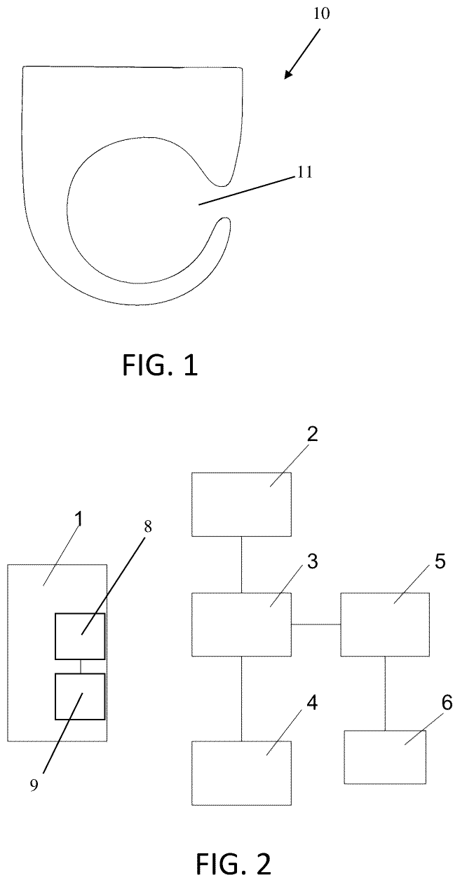

Referring to FIG. 2, The depicted apparatus comprises a smartphone (1) equipped with a microphone (8) for sound detection, a Bluetooth receiver module (2) seamlessly integrated into a flexible anti-snore ring (10), a microcontroller (3) responsible for device control, a vibration motor (4) for generating vibrations, a battery (5), and a battery charger (6) for recharging the battery.

To establish a connection between the smartphone (1) and the device, the user initiates a one-time download and installation of the related software application (9). The smartphone app (9) may allow users to customize the intensity, duration, and frequency of vibrations, as well as the length of snores, pauses between snores, and the number of snores to detect. This customization aims to enhance snore detection accuracy and user comfort. In this instance, the device takes the form of a wearable ring, worn on the user's finger, with the application launched on the smartphone before bedtime. The ring may define an adjustable ring opening (11) to accommodate fingers of varying sizes as shown in FIG. 1. In another version of the present invention, the snoring could be detected by means of a voice device like Alexa®.

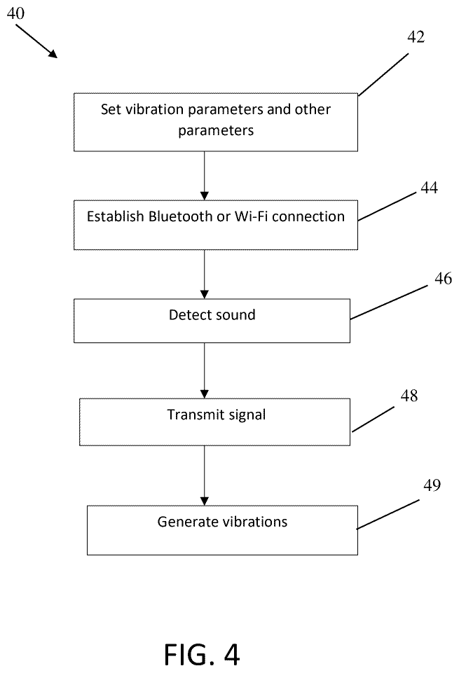

With reference to FIG. 4, an example methodology 40 for anti-snore detection and intervention is illustrated and described. While the methodology is described as being a series of acts or steps that are performed in a sequence, it is to be understood that the methodology is not limited by the order of the sequence. For instance, some acts or steps may occur in a different order than what is described herein. In addition, a step may occur concurrently with another step. Furthermore, in some instances, not all steps may be required to implement a methodology described herein.

Moreover, the steps or acts described herein may be computer-executable instructions that can be implemented by one or more processors and/or stored on a computer-readable medium or media. The computer-executable instructions may include a routine, a sub-routine, programs, a thread of execution, and/or the like. Still further, results of acts of the methodology may be stored in a computer-readable medium, displayed on the display device, and/or the like.

First, using the smartphone application, a user may set the intensity, duration, and frequency of vibrations generated by the anti-snore ring, and set the parameters for snore detection, including the length of snores, pauses between snores, and the number of snores to detect in step 42. Upon starting the application on the smartphone (1), it actively searches for and establishes a connection with the Bluetooth Low Energy (BLE) module or Wi-Fi module (2) in step 44. In step 46, the smartphone's microphone detects sounds, and the application analyses whether the detected sound corresponds to snoring. If snoring is identified, a Bluetooth signal is transmitted to the embedded Bluetooth module (2) in the ring in step 48. Subsequently, the Bluetooth module relays the signal to the microcontroller (3), which decodes it and directs the vibration motor (4) to initiate subtle vibrations in step 49. These vibrations prompt the user to adjust their position, addressing snoring without fully waking the individual.

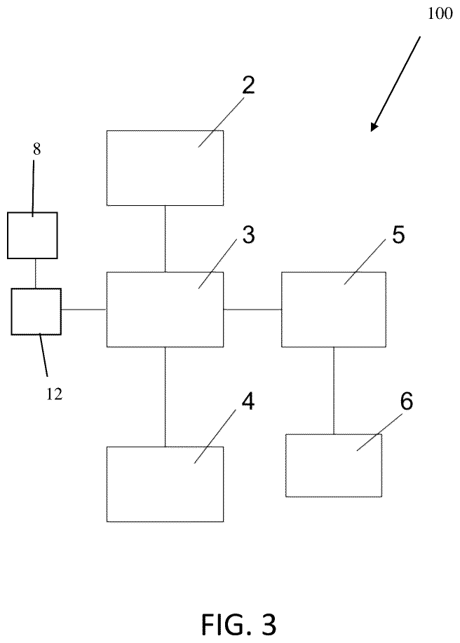

FIG. 3 shows another embodiment of the invention in which the device operates independently of a smartphone. In this embodiment the microphone is integrated with an anti- snore ring (100), featuring integrated microphone (8) and electronics for stand-alone snoring detection circuitry (12). The snore detection circuity is operative to detect snoring in response to the microphone detecting sound. The microcontroller is operative to receive and process signals from the snore detection circuity to operate the vibration motor to generate vibrations upon the snore detection circuity detecting snoring. All of the other components of this embodiment of FIG. 3 are similar in structure and function as that of the embodiment of FIGS. 1 and 2 of the present invention.

In both embodiments, the choice to position the anti-snore ring (10) on the finger offers increased sensitivity of the finger to tactile stimuli. The finger, being rich in nerve endings, is far more responsive to gentle vibrations, making the apparatus more effective at waking or alerting the user with minimal vibration strength. This heightened sensitivity allows for more efficient use of vibration as a prompt, leading to better outcomes in reducing snoring.

The flexible nature of the ring ensures that it maintains snug and consistent contact with the user's skin, allowing the vibrations to be felt more directly and effectively. Unlike rigid or fixed-size designs, which may not always provide optimal skin contact, the flexible ring adapts to the finger's shape and movement. This ensures that the vibration is transmitted more efficiently, even with a smaller vibration motor, thus enhancing the performance of the device.

The ring design enables the use of a smaller vibration motor, which is both lighter and more discreet. Because of the heightened sensitivity of the finger and the optimized contact achieved by the flexible fit, the device can rely on a smaller motor to produce effective vibrations. This not only improves user comfort by reducing the size and weight of the device, but also reduces the noise associated with larger motors, making it less intrusive during sleep.

By utilizing a smaller vibration motor and leveraging the finger's natural sensitivity, the device consumes significantly less power than larger, This leads to extended battery life and less frequent charging, making the product more convenient for users. The reduction in power consumption also aligns with the growing demand for energy-efficient wearable devices, making this invention more desirable in the marketplace.

The one-size-fits-all design of the flexible ring offers substantial benefits in terms of manufacturing, inventory management, and cost-efficiency. By eliminating the need for multiple sizes, the device reduces complexity in production and stocking. Manufacturers only need to create a single mold, lowering production costs and simplifying the supply chain. This also benefits retailers and consumers, as stocking and purchasing decisions are simplified, ensuring broader accessibility and availability of the product.

This embodiment demonstrates a seamless and user-friendly approach to snoring intervention, leveraging the connectivity between the smartphone and the anti-snore ring to detect and mitigate snoring disturbances during sleep.

The inconspicuous design of the ring allows users to address snoring discreetly, and the adjustable structure ensures comfort for diverse users. Bluetooth/Wi-Fi connectivity facilitates detailed insights into snoring habits, empowering effective sleep management. The unique features and functionalities outlined in the claims underscore the inventive nature of this wearable anti-snoring device. This invention offers a discreet, effective solution to improve sleep quality by mitigating snoring disturbances.

What is claimed is:

1. A method for anti-snore detection and intervention comprising:
a. detecting snoring sounds using a microphone integrated in a device;
b. analyzing the detected sounds to determine if snoring is present;
c. transmitting a signal to a wearable anti-snore ring if snoring is detected; and
d. generating vibrations in the wearable anti-snore ring for prompting the wearer of the wearable anti-snore ring to adjust their sleeping position in response to the wearable anti-snore ring receiving the transmitted signal when snoring is detected.

2. The method of claim 1, wherein the device is a smartphone, wherein transmitting a signal to a wearable anti-snore ring is performed via Bluetooth Low Energy (BLE) or Wi-Fi if snoring is detected, wherein the wearable anti-snore ring comprises:
a flexible ring structure made of silicon or similar material;
an adjustable opening to accommodate fingers of varying sizes;
a vibration motor for generating vibrations upon receiving the transmitted signal from the device; and
the Bluetooth Low Energy (BLE) or Wi-Fi module for receiving signals from the smartphone.

3. The method of claim 1, wherein detecting snoring sounds using a microphone integrated in a device includes using artificial intelligence (AI) software and algorithms to continuously learn and adapt to user-specific snoring behaviors, thereby enhancing detection accuracy of snoring over time.

4. The method of claim 1 further comprising:
d. prior to detecting snoring sounds using a microphone integrated in a device, setting the intensity, duration, and frequency of vibrations generated by the anti-snore ring; and
e. setting the parameters for snore detection, including the length of snores, pauses between snores, and the number of snores to detect.

5. The method of claim 4, wherein the device is a smartphone, wherein the microphone is integrated into the smartphone, wherein setting the intensity, duration, and frequency of vibrations generated by the anti-snore ring and setting the parameters for snore detection, including the length of snores, pauses between snores, and the number of snores to detects are performed by a smartphone application.

6. The method of claim 1, wherein the device is the anti-snoring ring.

7. An apparatus for anti-snore detection and intervention comprising:
a. a wearable anti-snore ring comprising:
a flexible ring structure;
a vibration motor;
a Bluetooth Low Energy (BLE) or Wi-Fi module; and
b. a smartphone comprising:
a microphone for sound detection;
a BLE or Wi-Fi transmitter;
a smartphone application, wherein the smartphone application is operative to analyze snoring and transmit signals.

8. The apparatus of claim 7, wherein the wearable anti-snore ring further comprises:
an adjustable opening to accommodate fingers of varying sizes; and
an integrated microcontroller, wherein the microcontroller is operative to receive and process signals from the smartphone and control the vibration motor.

9. The apparatus of claim 7, wherein the smartphone application comprises:
algorithms for analyzing sounds detected by the microphone to determine snoring;
a user interface for customizing parameters related to snore detection and vibration settings.

10. The apparatus of claim 7, further comprising a battery and a battery charger integrated into the wearable anti-snore ring for powering and recharging the device, respectively.

11. A wearable anti-snore apparatus for anti-snore detection and intervention comprising:
a flexible ring structure;
a vibration motor;
a microphone for sound detection;
a microcontroller embedded in the flexible ring structure; and
snore detection circuity, wherein the snore detection circuity is operative to detect snoring in response to the microphone detecting sound, wherein the microcontroller is operative to receive and process signals from the snore detection circuity to operate the vibration motor to generate vibrations upon the snore detection circuity detecting snoring.

12. A system for anti-snore detection and intervention comprising:
a. a wearable anti-snore ring comprising:
a flexible ring structure;
a vibration motor;
b. a smartphone comprising:
a microphone for sound detection;
a BLE or Wi-Fi transmitter;
a smartphone application, wherein the smartphone application is operative to analyze snoring and transmit signals; and
c. software applications running on both the wearable anti-snore ring and the smartphone for communication and control of the system.
